SEQUENCE LISTING (10 sequences)

SEQ ID NO: 1 - DNA, 35 bp, Artificial Sequence
  Synthetic Construct
ttaaataggtatcgcgtgcttactccggtggaccg

SEQ ID NO: 2 - DNA, 35 bp, Artificial Sequence
  Synthetic Construct
tcgcacttatctcgtaccgtgaacacctagcgcgt

SEQ ID NO: 3 - DNA, 35 bp, Artificial Sequence
  Synthetic Construct
gatcgacgtacatgcccgatccacggacattcttt

SEQ ID NO: 4 - DNA, 20 bp, Artificial Sequence
  Synthetic Construct
attggtacacgagccgagtc

SEQ ID NO: 5 - DNA, 51 bp, Artificial Sequence
  Synthetic Construct
actagatacccagcacgccttggacgcgacatatctcacccctggagcggc

SEQ ID NO: 6 - DNA, 51 bp, Artificial Sequence
  Synthetic Construct
gagctcacagttcctaaagtgtaatggccggctacgctaagcgcattgatt

SEQ ID NO: 7 - DNA, 51 bp, Artificial Sequence
  Synthetic Construct
caacgtgagctcttatcactcctatctgattccctatcgatacatgccagg

SEQ ID NO: 8 - DNA, 51 bp, Artificial Sequence
  Synthetic Construct
gtcataagtatactcctcacacggagtcgccacacctatgaagagtagtgt

SEQ ID NO: 9 - DNA, 51 bp, Artificial Sequence
  Synthetic Construct
ggtagcactagttgctccgagagggtctattgcggccgcagccggccatat

SEQ ID NO: 10 - DNA, 51 bp, Artificial Sequence
  Synthetic Construct
gttgtcgttgcgcctgaatctcgtataagatatacagggtgatcgacggct

(19) United States
(12) Patent Application Publication
(10) Pub. No.: US 2016/0369338 A1
(43) Pub. Date: Dec. 22, 2016

(54) PORTABLE DEVICE FOR NUCLEIC ACID-BASED AUTHENTICATION OF LIQUID SAMPLES

(71) Applicant: CertiRx, Inc., Research Triangle Park, NC (US)

(72) Inventor: Thomas J. Mercolino, Chapel Hill, NC (US)

(21) Appl. No.: 15/184,694

(22) Filed: Jun. 16, 2016

Related U.S. Application Data
(60) Provisional application No. 62/180,469, filed on Jun. 16, 2015.

(51) Int. Cl. C12Q 1/68

(52) U.S. Cl. CPC C12Q 1/6876

(57) ABSTRACT

Methods and portable detection systems are provided herein that allow for authentication of liquid samples that have been manufactured to include an authentication code consisting of two or more single-stranded nucleic acid markers. The authenticating nucleic acid can be detected without the need for sequencing. The methods provided herein address the problem of a lack of a simple way to verify the authenticity of liquid samples that may be of high-value or have crucial implications for safety at the point-of-use or point-of-sale. The ability to verify the authenticity at the point-of-use or point-of-sale can improve safety and prevent fraud for liquid samples including alcohol, wine, spirits, drinking water, perfume, vaccines, fuel, motor oil, brake fluid, and hydraulic fluid.

### CROSS REFERENCE TO RELATED APPLICATIONS

The presently disclosed subject matter claims the benefit of U.S. Provisional Patent Application Ser. No. 62/180,469, filed Jun. 16, 2015, the entire disclosure of which is incorporated herein by reference.

### TECHNICAL FIELD

This presently disclosed subject matter is directed to portable devices for nucleic acid-based authentication of liquid samples.

### BACKGROUND

Forgery, grey market, and illegal re-imports are considered an increasing issue in industry. There is currently no simple and inexpensive way to verify the authenticity of high-value liquid samples at the point-of-use or point-of-sale. Because of their intrinsic capability to carry diverse coded information, nucleic acids have been used to provide a secure, cost effective, and forensic method to help companies protect their intellectual property and brand. However, prior art techniques are not readily perceptible without the aid of special equipment. In addition, prior art techniques require a unique code, thereby necessitating that a unique target nucleic acid sequence be synthesized, increasing cost and decreasing practicality. Moreover, complex and expensive sequencing analysis has typically been required to identify such unique sequences. The presently disclosed subject matter provides methods and devices to allow for point-of-use or point-of-sale authentication of liquid samples without the drawbacks noted in the prior art.

### SUMMARY

In some embodiments, the presently disclosed subject matter is directed to a method of authenticating a liquid sample comprising an authentication code consisting of two or more single-stranded nucleic acid markers. Particularly, the method comprises applying a portion of the liquid sample to a sample zone of a lateral flow assay device having a membrane or a series of contiguous membranes in fluid communication and capable of supporting capillary flow of the liquid sample, wherein the membrane from proximal end to distal end comprises: i) the sample zone for receiving the portion of the liquid sample comprising the authentication code consisting of two or more single-stranded nucleic acid markers; ii) a reporter reagent; iii) a reaction zone having at least one immobilized capture oligonucleotide(s) corresponding to each of the two or more nucleic acid markers, wherein the immobilized capture oligonucleotide is complementary to at least a portion of each corresponding nucleic acid marker and capable of forming a duplex heterodimer therewith, the capture oligonucleotide(s) immobilized in a predetermined pattern that allows for detection of the duplex heterodimers after association of the reporter reagent therewith; and iv) a wicking zone to provide for the capillary flow of the liquid sample from the sample pad zone through the reaction pad zone; and detecting the reporter reagent, wherein detection of the reporter reagent in the predetermined pattern in the reaction zone indicates authentication of the liquid sample.

In some embodiments, the presently disclosed subject matter is directed to a lateral flow assay device is provided for authenticating a liquid sample comprising an authentication code consisting of two or more single-stranded nucleic acid markers, the device including: a membrane or a series of contiguous membranes in fluid communication and capable of supporting capillary flow of the liquid sample, wherein the membrane from proximal end to distal end comprises: i) a sample zone for receiving a portion of the liquid sample comprising the authentication code consisting of two or more single-stranded nucleic acid markers; ii) a reporter reagent; iii) a reaction zone having at least one immobilized capture oligonucleotide(s) corresponding to each of the two or more nucleic acid markers, wherein the immobilized capture oligonucleotide is complementary to at least a portion of each corresponding nucleic acid marker and capable of forming a duplex heterodimer therewith, the capture oligonucleotide(s) immobilized in a predetermined pattern that allows for detection of the duplex heterodimers after association of the reporter reagent therewith; and iv) a wicking zone to provide for the capillary flow of the liquid sample from the sample pad zone through the reaction pad zone.

In some embodiments, the presently disclosed subject matter is directed to a method of authenticating a liquid sample comprising an authentication code consisting of two or more single-stranded nucleic acid markers, the method comprising: dipping at least a portion of a dip stick assay device into the liquid sample, the dip stick assay device comprising: a membrane capable of supporting capillary flow of the liquid sample having a proximal end for holding the membrane and a distal end for dipping into the liquid sample, wherein the membrane has at least one immobilized capture oligonucleotide(s) at the distal end corresponding to each of the two or more single-stranded nucleic acid markers, wherein each immobilized capture oligonucleotide is complementary to at least a portion of each corresponding nucleic acid marker and capable of forming a duplex heterodimer therewith, and wherein the capture oligonucleotide(s) are immobilized in a predetermined pattern that allows for detection of the duplex heterodimers after association of a reporter reagent therewith; dipping at least a portion of the dip stick assay device into a solution comprising the reporter reagent; and detecting the reporter reagent, wherein detection of the reporter reagent in the predetermined pattern on the membrane indicates authentication of the liquid sample.

In some embodiments, the presently disclosed subject matter is directed to a dip stick assay device is provided for authenticating a liquid sample comprising an authentication code consisting of two or more single-stranded nucleic acid markers, the device comprising: i) a membrane capable of supporting capillary flow of the liquid sample having a proximal end for holding the membrane and a distal end for dipping into the liquid sample, wherein the membrane has at least one immobilized capture oligonucleotide(s) at the distal end corresponding to each of the two or more single-stranded nucleic acid markers, wherein each immobilized capture oligonucleotide is complementary to at least a portion of each corresponding nucleic acid marker and capable of forming a duplex heterodimer therewith, and wherein the capture oligonucleotide(s) are immobilized in a predetermined pattern that allows for detection of the duplex heterodimers after association of a reporter reagent therewith; and ii) the reporter reagent separately packaged.

### BRIEF DESCRIPTION OF THE DRAWINGS

FIG. 1a is a schematic diagram depicting a side view of a portable lateral flow detection system having four unique capture oligomers for verification of liquid samples according to one or more embodiments of the presently disclosed subject matter.

FIG. 1b is a schematic diagram depicting a top view of a portable dual lateral flow detection system having four capture oligomers divided among two lateral flow strips for verification of liquid samples according to one or more embodiments of the presently disclosed subject matter.

FIG. 2a is a schematic diagram depicting a side view of the portable lateral flow detection system of FIG. 1a and illustrates unbound dsDNA indicator antibody reporter reagent after application of a sample that does not contain authenticating nucleic acid markers according to one or more embodiments of the presently disclosed subject matter.

FIG. 2b is a schematic diagram depicting a side view of the portable lateral flow detection system of FIG. 1a and shows a dsDNA indicator antibody reporter reagent bound to the test area of the Reaction Zone after application of a sample that contains authenticating nucleic acid markers according to one or more embodiments of the presently disclosed subject matter.

FIG. 3a is a schematic diagram depicting a side view of the portable lateral flow detection system of FIG. 1a and illustrates a competition assay with a sample containing authenticating nucleic acid markers for verification of liquid samples according to one or more embodiments of the presently disclosed subject matter.

FIG. 3b is a schematic diagram depicting a side view of the portable lateral flow detection system of FIG. 1a and illustrates a competition assay performed on a sample that does not contain authenticating nucleic acid markers for verification of liquid samples according to one or more embodiments of the presently disclosed subject matter.

FIG. 4 is a schematic diagram depicting a portable dip stick format detection system and shows visualization/detection of four immobilized capture oligomers at the distal end of the dip stick after dipping it into a liquid sample containing authenticating nucleic acid markers and a reservoir containing a detector reagent for verification of liquid samples according to one or more embodiments of the presently disclosed subject matter.

### DETAILED DESCRIPTION

All publications cited below are hereby incorporated by reference. Unless defined otherwise, all technical and scientific terms used herein will have the commonly understood meaning to one of ordinary skill in the art to which the presently disclosed subject matter pertains.

It should be noted that as used herein and in the appended claims, the singular forms “a,” “an,” and “the” include plural reference unless the context clearly dictates otherwise. Thus, for example, a reference to “a population of entities” is a reference to one or more populations of entities and includes equivalents thereof known to those skilled in the art and so forth.

As used herein, the terms “oligomer”, “oligo”, “oligonucleotide”, “polynucleotide”, “nucleic acid”, “sDNA”, “sDNA oligo”, “ssDNA”, “ssDNA oligo”, and “single-stranded DNA” are used interchangeably.

As used herein, the terms “capture oligomer”, “capture oligo”, “capture sequence”, “capture oligonucleotide”, “capture polynucleotide”, “capture nucleic acid”, “capture DNA” are used interchangeably to refer to the capture nucleic acid molecule(s) located within the reaction zone of the lateral flow device or the distal end of the membrane of the dip stick device.

As used herein, the terms “single-stranded nucleic acid markers”, “nucleic acid markers”, “oligonucleotide markers”, and “polynucleotide markers” are used interchangeably to refer to the two or more single stranded nucleic acid marker molecules located within the sample.

The presently disclosed subject matter provides a simple way to verify the authenticity of liquid samples that may be of high-value or have crucial implications for safety at the point-of-use or point-of-sale. The liquid samples can include (but are not limited to) alcohol, wine, spirits, drinking water, perfume, vaccines, fuel, motor oil, brake fluid, and/or hydraulic fluid. The ability to verify the authenticity at the point-of-use or point-of-sale can improve safety and prevent fraud for liquid samples. Thus, methods and portable detection systems are provided herein to address this problem and allow for authentication of liquid samples that have been manufactured to include an authentication code consisting of two or more single-stranded nucleic acid markers. In the methods and portable detection systems provided herein, the authenticating nucleic acid can be detected without the need for sequencing of the nucleic acid markers.

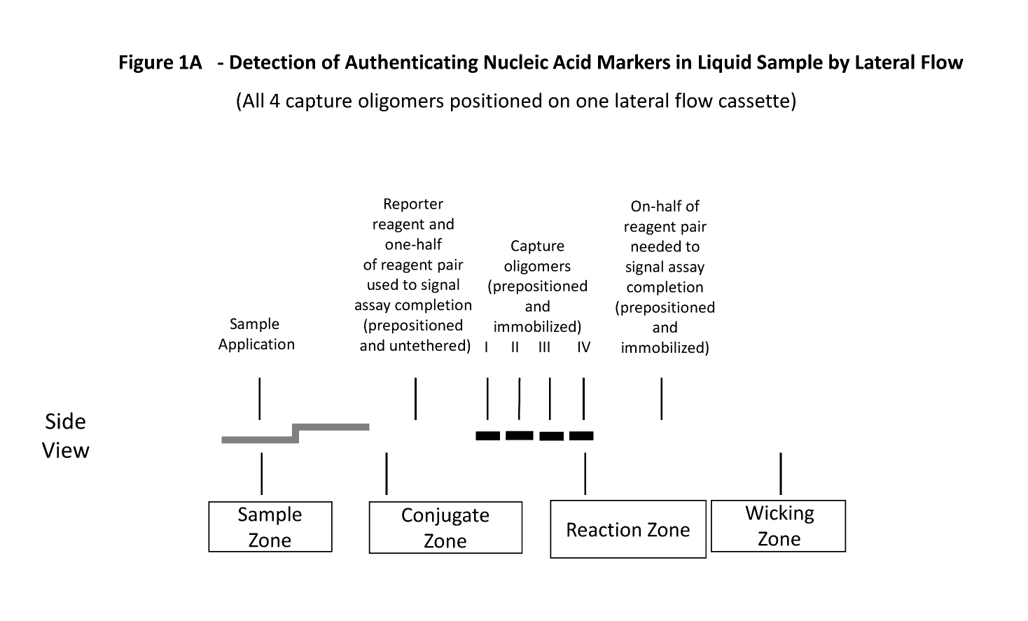

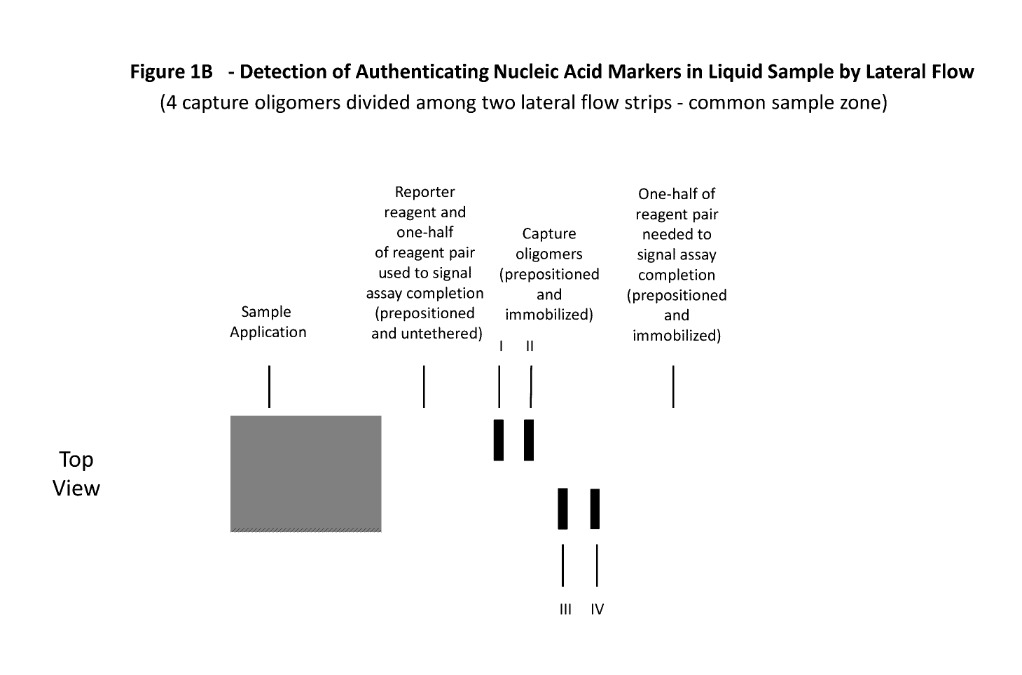

More specifically, in some embodiments, the presently disclosed subject matter comprises a method of authenticating a liquid sample comprising an authentication code consisting of two or more single-stranded nucleic acid markers. The method includes applying a portion of the liquid sample to the sample zone of a lateral flow assay device shown in FIGS. 1A-1B, 2A-2B, and 3A-3B. The method further includes detecting the authentication code in a predetermined pattern in a reaction zone of the lateral flow assay to indicate authentication of the liquid sample.

The lateral flow assay device shown in FIGS. 1A-1B, 2A-2B, and 3A-3B comprises a membrane or a series of contiguous membranes in fluid communication. The membrane or membranes are capable of supporting capillary flow of the liquid sample. Particularly, the membrane from proximal end to distal end comprises a sample zone, a reporter reagent, a reaction zone, and a wicking zone. The sample zone receives the portion of the liquid sample comprising the authentication code consisting of two or more single-stranded nucleic acid markers. The reporter reagent is capable of being mobilized by capillary flow of the liquid sample. The reaction zone comprises at least one immobilized capture oligonucleotide(s) corresponding to each of the two or more nucleic acid markers, wherein the immobilized capture oligonucleotide is complementary to at least a portion of each corresponding nucleic acid marker and is capable of forming a duplex heterodimer therewith. The capture oligonucleotide(s) are immobilized in a predetermined pattern that allows for detection of the duplex heterodimers after association of the reporter reagent therewith. The wicking zone provides for the capillary flow of the liquid sample from the sample pad zone through the reaction pad zone. Detection of the reporter reagent in the predetermined pattern in the reaction zone indicates authentication of the liquid sample.

In some embodiments, the lateral flow device can further include a conjugate zone, wherein the conjugate zone has a reporter reagent capable of being mobilized by capillary flow of the liquid sample.

The lateral flow systems of the present disclosure can be used to determine the presence of nucleic acids used as an authentication marker in an aqueous or organic solvent. Not to be limited to any particular method, a cartridge or cassette of membranes that creates a capillary flow or wicking action (much like that of the well-known home pregnancy test) can be exploited.

Upon receiving a liquid sample requiring authentication, a representative portion of the liquid sample containing one or more target nucleic acids can be applied to the lateral flow detection system of the present disclosure. In some embodiments, the sample can be applied by dropper or can be dispensed as a single dose, such as with a pre-calibrated disposable pipette. As the sample penetrates the sample zone and travels to the conjugate zone, it picks up one-half of a reagent pair required to signal assay completion. Simultaneously, the liquid sample picks up a DNA intercalating dye that moves with the sample-containing authentication sequence into the reaction zone. If the nucleic acid marker in the sample is complimentary to the sDNA prepositioned (striped) onto the reaction zone (capture oligomer), then a hybridization event occurs and the intercalating dye is immobilized by the newly formed dsDNA complex. The intercalating DNA dye is used to indicate the hybridization of the two complimentary strands. The dsDNA stain will have a stronger fluorescence when bound to dsDNA (typically 20× or more) and thus the area on the reaction zone will have a stronger fluorescence where the complimentary oligomer is prepositioned. Unbound DNA stain moves freely into the wicking zone (absorbent pad) when a sample does not contain the authenticating sequence and thus minimal fluorescence develops on top of the capture oligomer. The fluorescence can be determined qualitatively by passing a handheld light of suitable excitation frequency over the reaction zone (e.g., a UV light) and visualized by unaided vision or vision aided only by a filter, preferably one that passes the emitted fluorescence light but blocks much or most of the rest. Such blocking of light is typically achieved using a suitable long pass filter, a band-pass filter, or combinations thereof. Alternatively or in addition, the determination can be made by inserting the entire lateral flow device into any number of commercially available fluorescent readers adapted to accept the lateral flow device. In some embodiments, the portable reader can automatically advance the lateral flow test strip and stop the device when the fluorescence detector is immediately above a capture oligomer.

In some embodiments, the liquid sample comprising the authentication code consisting of two or more single-stranded nucleic acid markers penetrates the sample zone and is drawn by capillary action across a series of porous membranes (encompassing the conjugate zone, reaction zone, and wicking zone) that are forced into contact by a series of strategically placed pillars within a plastic housing.

In some embodiments, the lateral flow device can include a plurality of contiguous membranes (i.e., 3, 4, or 5) that can vary in pore size and chemical composition. In this configuration, the device will house a distinct “sample zone”, “conjugate zone”, “reaction zone”, and “wicking zone.” In some embodiments, the “sample zone” and “wicking zone” can be the same material. In some embodiments, the membrane or series of contiguous membranes can be supported by a backing or a housing. In some embodiments, the position reserved for sample application, conjugate, reaction and wicking is one continuous medium, as is the case for the FUSION 5 product developed by Millipore (Billerica, Mass.).

In some embodiments, the membrane comprising the reaction zone can be designed to detect more than one target nucleic acid sequence at a time. Specifically, the capture oligomers can be spaced apart on the reaction zone, but all within the path of sample flow. In some embodiments, four or more unique capture oligomers can be placed on the reaction zone of a single strip.

In some embodiments, the lateral flow device can comprise two separate lateral flow strips or series of contiguous membranes that can be used to detect an authentication code that consists of 2, 4 or more nucleic acid markers (see FIG. 1B). Each lateral flow strip can comprise a conjugate zone, reaction zone, and wicking zone. The reaction zone of each lateral flow strip can comprise one or more unique capture oligomers (see FIG. 1B). In some embodiments, the two lateral flow strips can comprise a common sample zone such that only one sample application is required. In these embodiments, the series of contiguous membranes can further include a second reaction zone carried by a second parallel membrane having a portion of the immobilized capture oligonucleotides.

The membrane of the reaction zone can have an immobilized capture oligomer that is about 100% complementary to the nucleic acid marker in the sample to be authenticated or it can intentionally have a lesser degree of homology. For example, the homology between capture and nucleic acid marker sDNA can be about 90%, 80%, 70% or some lesser degree (i.e., no more or no less than about 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, or 60% homology).

The reporter compound bound by the formation of the duplex heterodimer of the marker nucleic acid and the complementary capture oligomer can comprise any one of several DNA stains known and used in the art. For example, in some embodiments, the reporter can be an intercalating dsDNA stain, including (but not limited to) propidium iodide. In some embodiments, the reporter can comprise one or more minor grove dsDNA binders, including (but not limited to) DAPI and Hoechst stain. In some embodiments, bis-intercalating, major grove, and external dsDNA binders can also function as reporter stains. Thermo Fisher Scientific Corporation (Waltham, Mass.) provides an assortment of dsDNA binding stains. In some embodiments, the reporter stain increases in fluorescence intensity when bound to dsDNA. For example, propidium iodide, Hoechst stain, and DAPI excite at or around 348 nm. A portable lateral flow device fluorescence reader can be used to measure fluorescence, such as the ESE-Quant Lateral Flow Reader from ESE GmbH (Brunswick, Germany), FluoScan from ESE GmbH, and the FS-Scanner from Viewpoint (Prussia, Pa.).

In some embodiments, the sample zone and/or conjugate zone can comprise a pad from the Millipore Merck SureWick® Series. In some embodiments, the conjugate zone can be constructed from glass fiber (such as the Merck Millipore G041) or polyester. In some embodiments, the reaction zone can comprise a nitrocellulose pad, such as the Millipore Merck Hi-Flow™ Plus Series (240/180/135/120/90/75). Nylon and PVDF membranes can also serve as pads for the reaction zone. In some embodiments, the sample zone and/or wicking zone can comprise absorbent cellulose papers, such as the Millipore Merck C083. In some embodiments, the conjugate zone can comprise glass fiber and/or polyester. In some embodiments, the reaction zone can include nitrocellulose, nylon, and/or PVDF. In some embodiments, the sample zone and/or wicking zone can comprise cellulose paper.

The sample volume can typically be loaded in the range of about 40 to 120 μl for a single lateral flow device. In some embodiments, the sample volume loaded can be about 60 to 100 μl or 70 to 90 μl. Thus, the sample volume can be at least 40, 45, 50, 55, 60, 65, or 70 μl. In some embodiments, the sample volume can be at most 80, 85, 90, 95, 100, 105, 110, 115, 120, 125, or 130 μl. Greater or lesser sample volumes are also included within the scope of the presently disclosed subject matter.

Capture oligomer sequences can be spaced 3 to 10 millimeters apart, and can be adjusted to reduce crosstalk or to accommodate more capture oligomers within a single reaction zone. Thus, in some embodiments, the capture oligomer sequences can be spaced 3, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 millimeters apart.

A positive control DNA can be eluted from the conjugate zone and its binding to complementary sequence immobilized on the strip most distal thereto can be used to signal assay completion. Other non-DNA binding pairs can be used to signal assay completion, either in conjunction with positive control or alone, with one component of the pair immobilized downstream of the capture oligomer(s) and the other member of the pair prepositioned (but untethered) in the conjugate zone. For example, the binding pair can include (but is not limited to) avidin-biotin, whereby one member of the pair is fixed to a dye particle. In addition, the binding pair can include a peptide rarely found in nature and an antibody specific to the peptide, wherein one member of the peptide-antibody pair is fixed to a dye particle that is prepositioned but untethered in the conjugate zone and the other member of the pair is immobilized downstream of the capture oligomer(s).

The capture oligomer can be immobilized to the reaction zone membrane (nitrocellulose/nylon/PVDF) by methods known and used in the art, such as drying and/or baking. Short sDNA sequences can be challenging to absorb to reaction membranes and in certain embodiments require the addition of a Poly-T tail. Some membranes can be pretreated with poly-l-lysine before the oligomer will attach. On occasion when the sDNA is deformed upon immobilization to the reaction membrane it can lose its ability to bind the complementary DNA strand. In these situations, the capture sDNA can be biotinylated, combined with avidin, and then immobilized via capture of the DNA-hapten-protein complex to the reaction zone membrane. FIG. 1a illustrates one example of a lateral flow detection system involving four unique capture oligomers. Authentication can be conducted on a single membrane strip such as a nitrocellulose strip. FIG. 1b illustrates an example of a dual lateral flow assay system that can be contained in a common housing or shell, wherein each nitrocellulose strip contains two of four unique capture oligomers required for simultaneous authentication. As shown, the two lateral flow strips share a common sample zone. The housing that surrounds the membranes can have two distinct windows for viewing the fluorescence that can develop within each reaction zone.

In some embodiments, a single-stranded DNA containing the capture sequence can be immobilized to the reaction zone. In some embodiments, using longer length single-stranded DNA can overcome the challenges of absorbing short sDNA oligomer sequences. Moreover, the reaction zone membrane can serve to capture multiple different authentication codes that occur at different sequence positions of such longer length ssDNAs.

Upon receiving a liquid sample for authentication, a representative portion of the sample material containing the marker nucleic acids can be applied to a lateral flow device at the sample zone. As the sample penetrates the sample zone and travels into the conjugate zone, it can pick up a positive control DNA, one-half of a reagent pair required to signal assay completion, or both, as set forth above.

Simultaneously, the liquid sample can “pick up” an antibody specific to dsDNA (e.g., through solubilizing dried or lyophilized antibody), which then moves with the sample containing authentication sequence into the reaction zone. If the marker nucleic acid in the sample is complimentary to the capture oligomers prepositioned (striped) onto the reaction zone, then a hybridization event occurs and the dsDNA specific antibody will be immobilized by the newly formed dsDNA complex. The dsDNA specific antibody is used to indicate the hybridization of the two complimentary strands.

The binding of the dsDNA specific antibody causes a signal to develop in the area on the reaction zone where the complimentary capture oligomer is prepositioned. Unbound dsDNA specific antibody moves freely into the wicking zone (absorbent pad) when a sample does not contain the authenticating nucleic acid and thus minimal or no signal develops on top of the capture oligomer.

In some embodiments, the dsDNA specific antibody binder can be labeled with a small molecular weight fluorescent molecule such as Fluorescein Isothiocyanate (FITC). In embodiments where a signal enhancement is needed, the antibody can be conjugated to an enzyme (such as Horse Radish Peroxidase (HRP) or Alkaline Phosphatase (AP)). In some embodiments, even greater detection sensitivity is achieved when the dsDNA specific ligand is labeled with a particle (such as latex, gold or magnetite), or a sac containing dye (such as a liposome). Accordingly, the type of signal produced by a positive assay can be fluorescent, reflectant, absorbent and/or luminescent. In some embodiments, the assay can be colormetric and/or magnetic. The signal can be read visually or with the assistance of a portable instrumented reader.

In some embodiments, the indicator antibody selected for the disclosed assays has a high specificity for dsDNA and a low cross reactivity for ssDNA. An example of an antibody is the reagent from LifeSpan Biosciences Inc Cat # LS-C144515 from the hybridoma clone 11B6. Clone 11b6 produces an antibody that shows 100% reactivity with dsDNA and 5% reactivity with ssDNA. In some embodiments, the antibody recognizes short base pair sequences that are described frequently in the scientific literature.

In some embodiments, the disclosed method can include concentrating two or more single-stranded nucleic acid markers in the portion of the liquid sample prior to applying the portion of the liquid sample to the sample zone.

In some embodiments, the liquid sample can be an aqueous or an organic solvent. For example, the liquid sample can include (but is not limited to) alcohol, wine, spirits, drinking water, perfume, vaccines, fuel, motor oil, brake fluid, and/or hydraulic fluid.

In some embodiment, the liquid sample comprising the authentication code that includes two or more single-stranded nucleic acid markers can be the result of solubilization of the two or more single-stranded nucleic acid markers from a dried or lyophilized state. For example, such dried or lyophilized markers can serve as the authentication code for a solid article after application directly to the solid article. In some embodiments, dried or lyophilized markers can be mixed in dry form with other dry ingredients to serve as the authentication code for a non-liquid sample. In some embodiments, dried or lyophilized markers can be a component of an ink, a paint, or other coating to serve as the authentication code for these samples. In each of these examples, solubilization of the nucleic acid markers of the authentication code of the non-liquid sample results in a liquid sample comprising the authentication code for use with the methods and compositions of the presently disclosed subject matter. In some embodiments, the authentication code can consist of four single-stranded nucleic acid markers. The two or more single-stranded nucleic acid markers range in length from 10 to 60 nucleotides in some embodiments. The two or more single-stranded nucleic acid markers can be selected from the group consisting of a nucleic acid set forth in: TTA AAT AGG TAT CGC GTG CTT ACT CCG GTG GAC CG (SEQ ID NO: 1), TCG CAC TTA TCT CGT ACC GTG AAC ACC TAG CGC GT (SEQ ID NO: 2), GAT CGA CGT ACA TGC CCG ATC CAC GGA CAT TCT TT (SEQ ID NO: 3), ACG ATC ACG CGT GCA ATT GGT ACA CGA GCC GAG TC (SEQ ID NO: 4), ACTAGATACCCAGCACGCCTTGGACGCGACATATCTCACCCCTGGAGCGGC (SEQ ID NO: 5), GAGCTCACAGTTCCTAAAGTGTAATGGCCGGCTACGCTAAGCGCATTGATT (SEQ ID NO: 6), CAACGTGAGCTCTTATCACTCCTATCTGATTCCCTATCGATACATGCCAGG (SEQ ID NO: 7), GTCATAAGTATACTCCTCACACGGAGTCGCCACACCTATGAAGAGTAGTGT (SEQ ID NO: 8), GGTAGCACTAGTTGCTCCGAGAGGGTCTATTGCGGCCGCAGCCGGCCATAT (SEQ ID NO: 9), and GTTGTCGTTGCGCCTGAATCTCGTATAAGATATACAGGGTGATCGACGGCT (SEQ ID NO: 10).

In some embodiments, the two or more single-stranded nucleic acid markers can have a chemical modification at one or both of the 3′ or 5′ ends to prevent exonuclease degradation. The two or more single-stranded nucleic acid markers can be modified using any method known in the art to protect the nucleic acid markers from degradation and/or to improve solubility including, for example, the methods described in International Patent Publication No. WO 2013/143014, and/or the methods described in U.S. Patent Application Publication No. 2005/0008762 by Sheu, Jue-Jei. In the Sheu, Jue-Jei method, a water-insoluble medium comprising polymeric substances is first dissolved in an organic solvent to form a medium/solvent mixture. Then, nucleic acid marker solution in water, or suitable buffer, is mixed with an intermediate solution, such as methanol, ethanol, acetone, glycerol or their mixtures to form a homogenous mixture of nucleic acid marker solution. The two mixtures are then mixed to form a third homogenous mixture.

In some embodiments, the reporter reagent can comprise a DNA intercalating dye or a labeled dsDNA antibody. The DNA intercalating dye can be a fluorescent dye.

In some embodiments, the labeled dsDNA antibody can include a chromophore label, a fluorescent dye label, a conjugated enzyme, a conjugated Horse Radish Peroxidase (HRP) enzyme, a conjugated Alkaline Phosphatase (AP) enzyme, a latex particle label, a gold particle label, a magnetite label, or a sac having dye label, and/or a liposome having dye label.

In some embodiments, the reporter reagent can include a dsDNA intercalating dye, wherein one or more immobilized capture oligonucleotide is 50 to 59%, 60 to 69%, 70 to 79%, 80 to 89%, or 90 to 99% complementary to the portion of the corresponding nucleic acid marker, and wherein the corresponding predetermined change in detection of the dsDNA intercalating dye indicates authentication of the liquid sample.

In some embodiments, the lateral flow assay device can further include: in the conjugate zone, a first half of a reagent pair; and in the reaction zone, a second half of the reagent pair capable of binding to the first half of the reagent pair to produce a bound reagent pair, wherein the second half of the reagent pair is immobilized downstream of the immobilized capture oligonucleotide(s), wherein association of the reporter reagent or a second reporter molecule with the bound reagent pair allows for detection of the bound reagent pair to indicate assay completion; and the method can optionally further include detecting the second reporter molecule to indicate assay completion.

In some embodiments, the first and second halves of the reagent pair can include single-stranded complementary oliognucleotides capable of forming a duplex heterodimer, and the bound reagent pair can be the duplex heterodimer.

In some embodiments, the first half of the reagent pair can include an avidin or a biotin having the second reporter molecule attached thereto, and the second half of the reagent pair can include a corresponding avidin or biotin, wherein detection of the second reporter molecule immobilized in the reaction zone indicates assay completion.

In some embodiments, the first half of the reagent pair can include a peptide having the second reporter molecule attached thereto, and the second half of the reagent pair can include an antibody specific to the peptide, wherein detection of the second reporter molecule immobilized in the reaction zone indicates association of the reagent pair and assay completion.

In some embodiments, detecting the reporter reagent can be carried out using a handheld light of suitable excitation frequency or by inserting the device into a fluorescent reader. The reporter reagent can be a chromophore, and detecting the reporter reagent can be carried out by non-instrumented visual inspection of a color change resulting from the signal produced by the chromophore.

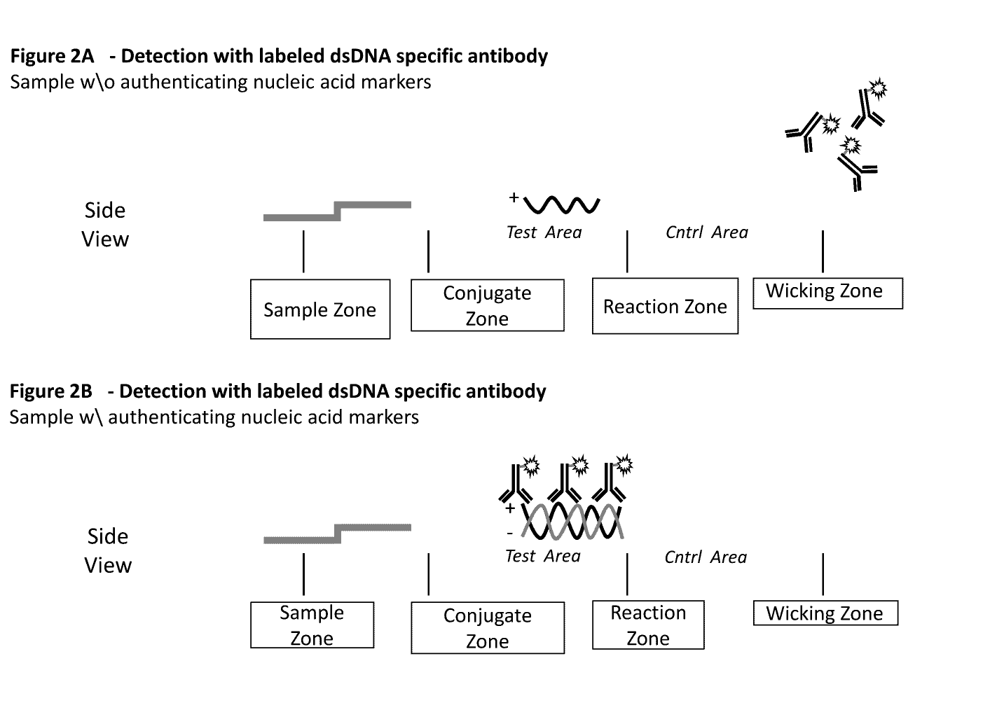

FIG. 2a illustrates the movement of the dsDNA indicator antibody reagent in a sample that does not contain authenticating nucleic acid marker. Particularly, FIG. 2a is a schematic diagram depicting a side view of the portable lateral flow detection system of FIG. 1a and illustrates unbound dsDNA indicator antibody reporter reagent ( [symbol] ) after application of a sample that does not contain authenticating nucleic acid markers for verification of liquid samples according to one or more embodiments of the present disclosure.

FIG. 2b illustrates shows the movement of the dsDNA indicator antibody reagent in a sample that contains the authenticating nucleic acid marker. Specifically, FIG. 2b is a schematic diagram depicting a side view of the portable lateral flow detection system of FIG. 1a and shows a dsDNA indicator antibody reporter reagent ( [symbol] ) bound to the test area of the Reaction Zone after application of a sample that contains authenticating nucleic acid markers for verification of liquid samples according to one or more embodiments of the present disclosure. As described previously, a labeled dsDNA detector antibody can be prepositioned in the conjugate zone before sample is loaded.

Using non-labeled nucleic acid markers to compete with labeled nucleic acid markers is the assay configuration which can deliver a high level of precision and reproducibility. In this format, higher quantities of authenticating nucleic acid markers in the sample translates into less signal on the lateral flow reaction zone. A sample that does not contain authenticating nucleic acid markers will not lead to a reduction in signal on the reaction zone (nitrocellulose, nylon, etc.). The labeled nucleic acid marker can be prepositioned in the conjugate zone or combined with sample before the sample is loaded onto the sample zone. Thus, in some embodiments, the lateral flow assay device can further include in the conjugate zone the two or more single-stranded nucleic acid markers having the reporter reagent covalently attached, wherein the single-stranded nucleic acid markers are capable of being mobilized by capillary flow of the liquid sample, and wherein the assay is a competition assay wherein decreased detection of the reporter reagent indicates the presence of the authentication code. While a competitive lateral flow device result can be read visually in certain embodiments, the combination of a competitive lateral flow device assay configuration with an instrumented read is preferred.

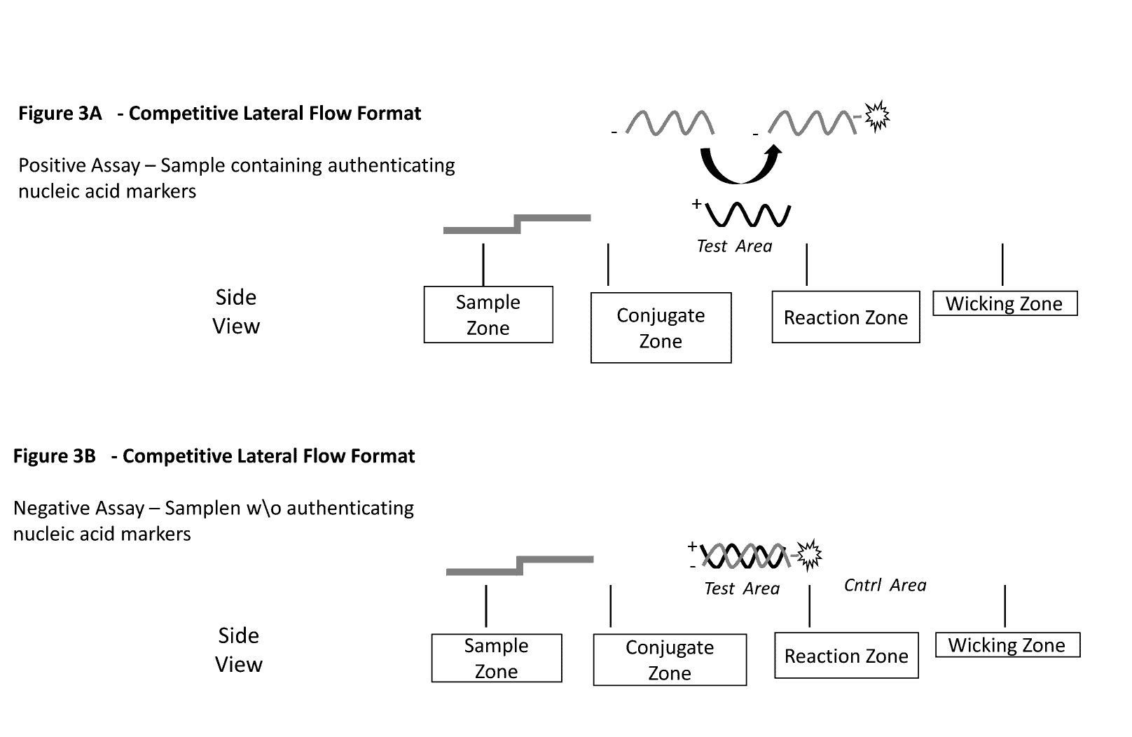

FIG. 3a is a schematic diagram depicting a side view of the lateral flow detection system of FIG. 1a and illustrates a competition assay with a sample containing authenticating nucleic acid markers for verification of liquid samples according to one or more embodiments of the present disclosure. FIG. 3b is a schematic diagram depicting a side view of the lateral flow detection system of FIG. 1a and illustrates a competition assay performed on a sample that does not contain authenticating nucleic acid markers for verification of liquid samples according to one or more embodiments of the present disclosure.

In some embodiments, disclosed device can be assembled in a lateral flow device or can be assembled in a dip stick format. Dip stick nucleic acid hybridization assays are known in the art and can be found in U.S. Pat. Nos. 7,186,508 and 5,310,650.

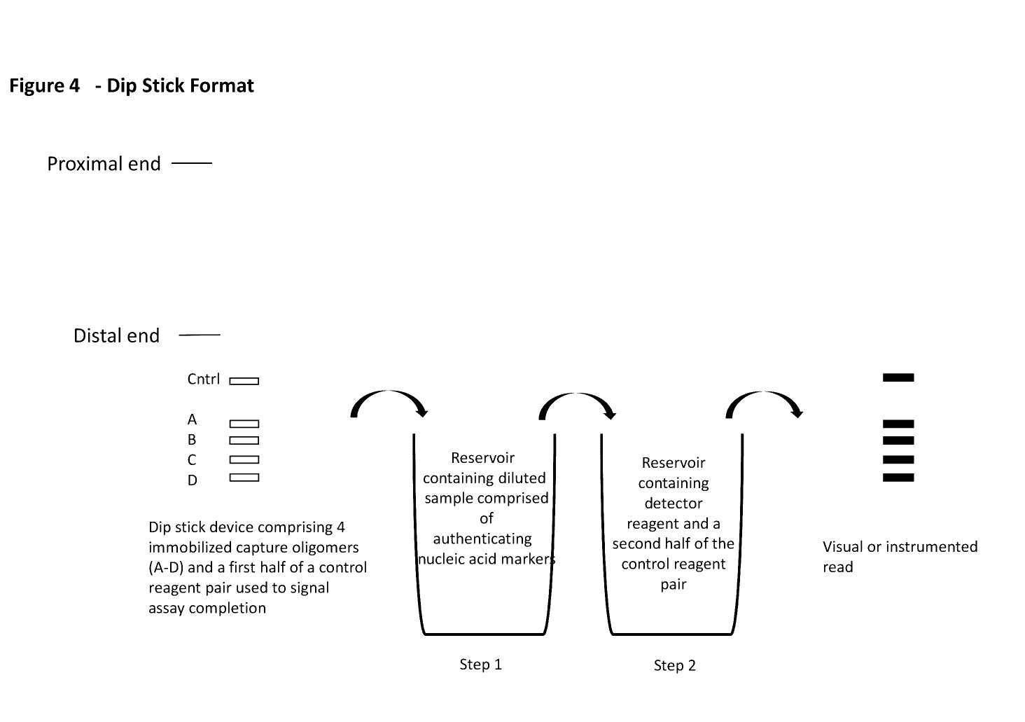

In some embodiments, the presently disclosed subject matter is directed to a method for authenticating a liquid sample comprising an authentication code consisting of two or more single-stranded nucleic acid markers that includes dipping at least a portion of a dip stick assay device shown in FIG. 4 into the liquid sample, and detecting the authentication code in a predetermined pattern on a membrane of the dip stick assay device to indicate authentication of the liquid sample.

The dip stick assay device includes: a membrane capable of supporting capillary flow of the liquid sample having a proximal end for holding the membrane and a distal end for dipping into the liquid sample, wherein the membrane has at least one immobilized capture oligonucleotide(s) at the distal end corresponding to each of the two or more single-stranded nucleic acid markers, wherein each immobilized capture oligonucleotide is complementary to at least a portion of each corresponding nucleic acid marker and capable of forming a duplex heterodimer therewith, and wherein the capture oligonucleotide(s) are immobilized in a predetermined pattern that allows for detection of the duplex heterodimers after association of a reporter reagent therewith (see FIG. 4). Detection of the reporter reagent in the predetermined pattern on the membrane indicates authentication of the liquid sample.

Similar to the lateral flow device configuration of the present disclosure, a dip stick device configuration of the present disclosure can include a membrane capable of supporting capillary flow of the liquid sample and having a proximal end for holding the membrane and a distal end for dipping into the liquid sample. The membrane can be supported by a backing or a housing. The membrane can include nitrocellulose, nylon, or PVDF. The distal end can consist of nylon, nitrocellulose, or an equivalent. The distal end can be treated to comprise immobilized capture oligomer and one-half of a control reagent pair used to signal assay completion. The dip stick device comprising immobilized capture oligomer can be treated further with a substance to reduce non-specific binding, then dried and stored away until needed.

As shown in FIG. 4, Step 1, when a sample has been selected for verification, the distal end of a dip stick strip device can be dipped into a reservoir containing a representative volume of the sample. If the sample contains nucleic acid markers, hybridization events occur with immobilized capture oligomer to form dsDNA on the surface of the dipstick device. In some embodiments, the entire distal end of the dip stick device is not submerged and, rather, only a small portion of the distal end makes contact with the liquid sample and can be held in place until the sample volume has wicked upward and beyond the area where control reagent was immobilized. The first half of the control reagent can be biotinylated bovine serum albumin. Certain samples may require the entire distal end to be submerged during Step 1.

In Step 2 of FIG. 4, the distal end of the dip stick device is placed in contact with a solution containing detector reagent and a second half of the control reagent. The detector reagent can be a particle coated with antibody specific to dsDNA. The second half of the control reagent can be a detectable particle coated with avidin. For speed it is more beneficial to submerge the entire reaction pad into the Step 2 solution.

The dipstick format is not restricted to the use of an indicator particle coated with antibody specific for dsDNA. Other indicator reagents can be substituted in alternative configurations.

As in the lateral flow device configuration, the proximal and distal ends of the dip stick device can be supported on one side by a backer (e.g., a plastic strip with adhesive) that holds the proximal and distal ends in place.

One or more wash steps can be performed after Step 1 and Step 2 being mindful of the stringency so not to remove capture oligomer or dsDNA or control reagents used to signal assay completion.

While four distinct capture oligomers are modeled in FIG. 4, the dip stick device format is less reliant on capillary flow and additional capture oligomers can be deployed.

Thus, in some embodiments, a method is provided for authenticating a liquid sample having an authentication code consisting of two or more single-stranded nucleic acid markers, that includes: dipping at least a portion of a dip stick assay device into the liquid sample, the dip stick assay device including: a membrane capable of supporting capillary flow of the liquid sample having a proximal end for holding the membrane and a distal end for dipping into the liquid sample, wherein the membrane has at least one immobilized capture oligonucleotide(s) at the distal end corresponding to each of the two or more single-stranded nucleic acid markers, wherein each immobilized capture oligonucleotide is complementary to at least a portion of each corresponding nucleic acid marker and capable of forming a duplex heterodimer therewith, and wherein the capture oligonucleotide(s) are immobilized in a predetermined pattern that allows for detection of the duplex heterodimers after association of a reporter reagent therewith; dipping at least a portion of the dip stick assay device into a solution comprising the reporter reagent; and detecting the reporter reagent, wherein detection of the reporter reagent in the predetermined pattern on the membrane indicates authentication of the liquid sample.

In some embodiments, the membrane of the dip stick device can further include a first half of a reagent pair immobilized on the membrane at a predetermined position proximal to the immobilized capture oligonucleotide(s), and the method can further include dipping the at least a portion of the dip stick assay device into a solution comprising a second half of the reagent pair capable of binding to the first half of the reagent pair to produce a bound reagent pair, wherein association of the reporter reagent or a second reporter molecule with the bound reagent pair allows for detection of the bound reagent pair to indicate assay completion, and wherein the method optionally can further include detecting the second reporter molecule to indicate assay completion.

In some embodiments, the first and second halves of the reagent pair can include single-stranded complementary oliognucleotides capable of forming a duplex heterodimer, and the bound reagent pair can be the duplex heterodimer. The first half of the reagent pair can include an avidin or a biotin, and the second half of the reagent pair can include a corresponding avidin or biotin having the second reporter molecule attached thereto, wherein detection of the second reporter molecule immobilized on the membrane indicates assay completion.

In some embodiments, the first half of the reagent pair can include a peptide or protein, and the second half of the reagent pair can include an antibody specific to the peptide or protein having the second reporter molecule attached thereto, wherein detection of the second reporter molecule immobilized on the membrane indicates assay completion.

In some embodiments, the detection can be carried out using a handheld UV light or by inserting the dip stick device into a fluorescent reader. The reporter reagent can be a chromophore, and detection can be carried out by non-instrumented visual inspection of a color change resulting from the signal produced by the chromophore.

### REFERENCES

1. http://scitechconnect.elsevier.com/wp-content/uploads/2014/03/main

Chapter 24, Lateral Flow Immunoassay Systems: Evolution from the Current State of the Art to the Next Generation of Highly Sensitive, Quantitative Rapid Assays
2. EMD Millipore Corporation Technical Guide (2013)

Rapid Lateral Flow Test Strips Considerations for Product Development
3. Royal Society of Chemistry (2010)

Fang et al., Lateral Flow Nucleic Acid Biosensor for Cu²⁺Detection in Aqueous Solution with High Sensitivity and Selectivity
4. IVD Technology (2001)

Jones et al., Membrane immobilization of nucleic acids, Part 2, Probe Attachment Techniques
5. U.S. Pat. No. 7,186,508
6. U.S. Pat. No. 5,310,650
7. U.S. Patent Application 2012/0107956
8. International Patent Publication No. WO 2013/143014

What is claimed is:

1. A lateral flow assay device for authenticating a liquid sample comprising an authentication code consisting of two or more single-stranded nucleic acid markers, the device comprising:
a membrane or a series of contiguous membranes in fluid communication and capable of supporting capillary flow of the liquid sample, wherein the membrane from proximal end to distal end comprises:
i) a sample zone for receiving a portion of the liquid sample comprising the authentication code consisting of two or more single-stranded nucleic acid markers;
ii) a reporter regent;
iii) a reaction zone having at least one immobilized capture oligonucleotide(s) corresponding to each of the two or more nucleic acid markers, wherein the immobilized capture oligonucleotide is complementary to at least a portion of each corresponding nucleic acid marker and capable of forming a duplex heterodimer therewith, the capture oligonucleotide(s) immobilized in a predetermined pattern that allows for detection of the duplex heterodimers after association of the reporter reagent therewith; and
iv) a wicking zone to provide for the capillary flow of the liquid sample from the sample pad zone through the reaction pad zone.

2. The lateral flow device of claim 1 further comprising a conjugate zone, wherein the conjugate zone comprises the reporter reagent capable of being mobilized by capillary flow of the liquid sample.

3. The lateral flow device of claim 1, further comprising a concentrating component to concentrate the two or more single-stranded nucleic acid markers in the liquid sample prior to the liquid sample being received on the sample zone.

4. The lateral flow assay device of claim 1, wherein the liquid sample comprises an aqueous or an organic solvent.

5. The lateral flow assay device of claim 1, wherein the authentication code comprises four single-stranded nucleic acid markers.

6. The lateral flow assay device of claim 1, wherein the two or more single-stranded nucleic acid markers have a chemical modification at one or both of the 3′ or 5′ ends to prevent exonuclease degradation.

7. The lateral flow assay device of claim 1, wherein the two or more single-stranded nucleic acid markers are selected from the group consisting of a nucleic acid set forth in: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10.

8. The lateral flow assay device of claim 1, wherein the reporter reagent comprises a DNA intercalating dye or a labeled dsDNA antibody.

9. The lateral flow device of claim 2, further comprising:
in the conjugate zone, a first half of a reagent pair; and
in the reaction zone, a second half of the reagent pair capable of binding to the first half of the reagent pair to produce a bound reagent pair, wherein the second half of the reagent pair is immobilized downstream of the immobilized capture oligonucleotide(s),
wherein association of the reporter reagent or a second reporter molecule with the bound reagent pair allows for detection of the bound reagent pair to indicate assay completion.

10. A method for authenticating a liquid sample comprising an authentication code consisting of two or more single-stranded nucleic acid markers, the method comprising:
applying a portion of the liquid sample to a sample zone of a lateral flow assay device having a membrane or a series of contiguous membranes in fluid communication and capable of supporting capillary flow of the liquid sample, wherein the membrane from proximal end to distal end comprises:
i) the sample zone for receiving the portion of the liquid sample comprising the authentication code consisting of two or more single-stranded nucleic acid markers;
ii) a reporter reagent;
iii) a reaction zone having at least one immobilized capture oligonucleotide(s) corresponding to each of the two or more nucleic acid markers, wherein the immobilized capture oligonucleotide(s) is complementary to at least a portion of each corresponding nucleic acid marker and capable of forming a duplex heterodimer therewith, the capture oligonucleotide(s) immobilized in a predetermined pattern that allows for detection of the duplex heterodimers after association of the reporter reagent therewith; and
iv) a wicking zone to provide for the capillary flow of the liquid sample from the sample pad zone through the reaction pad zone; and
detecting the reporter reagent, wherein detection of the reporter reagent in the predetermined pattern in the reaction zone indicates authentication of the liquid sample.

11. A dip stick assay device for authenticating a liquid sample comprising an authentication code consisting of two or more single-stranded nucleic acid markers, the device comprising:
i) a membrane capable of supporting capillary flow of the liquid sample having a proximal end for holding the membrane and a distal end for dipping into the liquid sample, wherein the membrane has at least one immobilized capture oligonucleotide(s) at the distal end corresponding to each of the two or more single-stranded nucleic acid markers, wherein each immobilized capture oligonucleotide is complementary to at least a portion of each corresponding nucleic acid marker and capable of forming a duplex heterodimer therewith, and wherein the capture oligonucleotide(s) are immobilized in a predetermined pattern that allows for detection of the duplex heterodimers after association of a reporter reagent therewith; and
ii) the reporter reagent separately packaged.

12. The dip stick assay device of claim 11, wherein the authentication code comprises four or more single-stranded nucleic acid markers.

13. The dip stick assay device of claim 11, wherein the two or more single-stranded nucleic acid markers have a chemical modification at one or both of the 3′ or 5′ ends to prevent exonuclease degradation.

14. The dip stick assay device of claim 11, wherein the two or more single-stranded nucleic acid markers are selected from the group consisting of a nucleic acid set forth in: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10.

15. The dip stick assay device of claim 11, wherein the reporter reagent comprises a DNA intercalating dye or a labeled dsDNA antibody.

16. The dip stick assay device of claim 11, further comprising:
a first half of a reagent pair immobilized on the membrane at a predetermined position proximal to the immobilized capture oligonucleotide(s); and
a separately packaged second half of the reagent pair capable of binding to the first half of the reagent pair to produce a bound reagent pair, wherein association of the reporter reagent or a second reporter molecule with the bound reagent pair allows for detection of the bound reagent pair to indicate assay completion.

17. A method for authenticating a liquid sample comprising an authentication code consisting of two or more single-stranded nucleic acid markers, the method comprising:
dipping at least a portion of a dip stick assay device into the liquid sample, the dip stick assay device comprising:
a membrane capable of supporting capillary flow of the liquid sample having a proximal end for holding the membrane and a distal end for dipping into the liquid sample, wherein the membrane has at least one immobilized capture oligonucleotide(s) at the distal end corresponding to each of the two or more single-stranded nucleic acid markers, wherein each immobilized capture oligonucleotide is complementary to at least a portion of each corresponding nucleic acid marker and capable of forming a duplex heterodimer therewith, and wherein the capture oligonucleotide(s) are immobilized in a predetermined pattern that allows for detection of the duplex heterodimers after association of a reporter reagent therewith;
dipping at least a portion of the dip stick assay device into a solution comprising the reporter reagent; and
detecting the reporter reagent, wherein detection of the reporter reagent in the predetermined pattern on the membrane indicates authentication of the liquid sample.

18. The method of claim 17, wherein the liquid sample comprises alcohol, wine, spirits, drinking water, perfume, vaccines, fuel, motor oil, brake fluid, or hydraulic fluid.
